(19) United States
(12) Patent Application Publication
(10) Pub. No.: US 2023/0304086 A1
(43) Pub. Date: Sep. 28, 2023

(54) LABELED AVIDIN AND METHODS FOR SEQUENCING

(71) Applicant: Illumina Cambridge Limited, Cambridge (GB)

(72) Inventors: Xiaolin WU, Cambridge (GB); Patrick MCCAULEY, Cambridge (US); Madushani DHARMARWARDANA, Cambridge (GB)

(21) Appl. No.: 18/190,330

(22) Filed: Mar. 27, 2023

Related U.S. Application Data
(60) Provisional application No. 63/324,564, filed on Mar. 28, 2022.

(51) Int. Cl. C12Q 1/6869; C12Q 1/6876; C07K 14/36

(52) U.S. Cl. CPC C12Q 1/6869; C12Q 1/6876; C07K 14/36; C07K 2319/22

(57) ABSTRACT

Embodiments of the present disclosure relate to compositions and methods for improving the intensity of the fluorescent signals during nucleic acid sequencing. In particular, at least one biotin-binding site of the labeled streptavidin is blocked to reduce fluorescent signal deflation.

### CROSS-REFERENCE TO RELATED APPLICATIONS

This application claims priority to U.S. Provisional Application No. 63/324,564, filed Mar. 28, 2022, the content of which is incorporated by reference in its entirety.

### FIELD

The present disclosure relates to labeled avidin molecule for use in various biological applications, such as nucleic acid sequencing applications.

### BACKGROUND

Non-radioactive detection of nucleic acids utilizing fluorescent labels is an important technology in molecular biology. Many procedures employed in recombinant DNA technology previously relied on the use of nucleotides or polynucleotides radioactively labeled with, for example ³²P. Radioactive compounds permit sensitive detection of nucleic acids and other molecules of interest. However, there are serious limitations in the use of radioactive isotopes such as their expense, limited shelf life and more importantly safety considerations. Eliminating the need for radioactive labels enhances safety whilst reducing the environmental impact and costs associated with, for example, reagent disposal. Methods amenable to non-radioactive fluorescent detection include by way of non-limiting example, automated DNA sequencing, hybridization methods, real-time detection of polymerase-chain-reaction products and immunoassays.

For many applications it is desirable to employ multiple spectrally distinguishable fluorescent labels in order to achieve independent detection of a plurality of spatially overlapping analytes. In such multiplex methods the number of reaction vessels may be reduced to simplify experimental protocols and facilitate the production of application-specific reagent kits. In multi-color automated DNA sequencing systems for example, multiplex fluorescent detection allows for the analysis of multiple nucleotide bases in a single electrophoresis lane, thereby increasing throughput over single-color methods, and reducing uncertainties associated with inter-lane electrophoretic mobility variations.

However, multiplex fluorescent detection can be problematic and there are a number of important factors that may constrain selection of appropriate fluorescent labels. First, it may be difficult to find dye compounds with suitably-resolved absorption and emission spectra in a given application. In addition, when several fluorescent dyes are used together, generating fluorescence signals in distinguishable spectral regions by simultaneous excitation may be complicated because absorption bands of the dyes are usually widely separated, so it may be difficult to achieve comparable fluorescence excitation efficiencies even for two dyes. Another consideration of particular importance to molecular biology methods is the extent to which the fluorescent dyes must be compatible with reagent chemistries such as, for example, DNA synthesis solvents and reagents, buffers, polymerase enzymes, and ligase enzymes. Further, since many excitation methods use high power light sources like lasers, the fluorescent dyes must be sufficiently photo-stable to withstand multiple excitations. Strong fluorescence signals are especially important when measurements are made in water-based biological buffers and at higher temperatures as the fluorescence intensities of most organic dyes are significantly lower under such conditions.

Some optical and technical developments have already led to greatly improved image quality but were ultimately limited by poor optical resolution. Generally, optical resolution of light microscopy is limited to objects spaced at approximately half of the wavelength of the light used. In practical terms, then, only objects that are laying quite far apart (at least 200 to 350 nm) could be resolved by light microscopy. One way to improve image resolution and increase the number of resolvable objects per unit of surface area is to use excitation light of a shorter wavelength. For example, if light wavelength is shortened by Δλ˜100 nm with the same optics, resolution will be better (about Δ50 nm/(about 15%)), less-distorted images will be recorded, and the density of objects on the recognizable area will be increased about 35%.

Certain nucleic acid sequencing methods employ laser light to excite and detect dye-labeled nucleotides. To detect more densely packed nucleic acid sequencing clusters while maintaining useful resolution, a shorter wavelength blue light source may be used. However, a light source with a shorter wavelength may bleach fluorescent dyes and/or damage nucleotide samples in solution/on flow-cell surface or those to which the fluorescent dyes are conjugated. In addition, dye-dye quenching or dye self-quenching have also been observed. All of which may cause fluorescence signal intensity decay observed in the array context.

As such, there exists a need for developing new sequencing labeling reagents to improve fluorescent signal and intensity in SBS with more densely packed clusters, facilitating longer read length, as well as improving the quality of the sequencing results. Described herein are labeled avidin molecules for nucleic acid sequencing.

### SUMMARY

One aspect of the present disclosure relates to a labeled avidin for polynucleotide sequencing, comprising an avidin labeled with one or more detectable labels (e.g., fluorescent dye moieties), wherein at least one biotin-binding site of the avidin is blocked with a biotin moiety-containing molecule or an analog thereof, and wherein the labeled avidin comprises at least one open biotin-binding site. In some embodiments, one, two, or three biotin-binding sites of the avidin are blocked with the biotin moiety-containing molecule or the analog thereof. In some embodiments, the avidin is labeled with 2, 3, 4, 5, 6, 7, or 8 fluorescent dye moieties. In one embodiment, the avidin is labeled with six fluorescent dye moieties. In some such embodiments, the fluorescent dye moieties are identical or spectrally indistinguishable. In other embodiments, the fluorescent dye moieties are spectrally distinguishable. In some embodiments, the fluorescent dye moieties are excitable by a light source having a wavelength between 400 nm to 650 nm. In further embodiments, the fluorescent dye moieties are excitable by a light source having a wavelength between about 450 nm to about 460 nm, or between about 520 nm to about 535 nm. In some embodiments of the avidin described herein, the avidin is streptavidin. In some embodiments, the biotin moiety-containing molecule or the analog thereof comprises a biotin moiety that is covalently attached to a nucleotide or nucleoside. In some other embodiments, the biotin moiety-containing molecule or the analog thereof is a free biotin molecule or an analog that is not attached to another compound (e.g., the biotin moiety is not covalently attached to a nucleotide or nucleoside).

One aspect of the present disclosure relates to a labeled avidin nucleotide conjugate, comprising an avidin-binding partner covalently attached to the nucleotide, optionally via a cleavable linker, wherein the avidin-binding partner is conjugated with the labeled avidin described herein via non-covalent interaction. In some embodiments, the avidin-binding partner comprise a biotin moiety. In further embodiments, the avidin-binding partner is covalently attached to the nucleobase of the nucleotide via a cleavable linker. In some embodiments, the cleavable linker comprising an azido, allyl, or disulfide moiety. In further embodiments, the nucleotide conjugate comprises a 3′ blocking group. The nucleotide may be a nucleotide triphosphate comprising a 2′ deoxyribose.

Another aspect of the present disclosure relates to an oligonucleotide or polynucleotide conjugate, comprising an avidin conjugated to a terminal nucleotide moiety of the oligonucleotide or polynucleotide, wherein the avidin is labeled with one or more detectable labels (e.g., fluorescent dye moieties), and at least one biotin-binding site of the avidin is blocked with a biotin moiety-containing molecule or an analog thereof, wherein the terminal nucleotide moiety comprises an avidin binding partner covalently attached thereto, and the labeled avidin is conjugated to the avidin binding partner of the terminal nucleotide moiety by non-covalent interaction. In some embodiments, one, two or three biotin-binding sites of the avidin is blocked with the biotin moiety-containing molecule or the analog thereof. In some embodiments, the labeled avidin comprises 2, 3, 4, 5, 6, 7, or 8 dye moieties. In some embodiments, the biotin moiety-containing molecule or the analog thereof comprises a biotin moiety covalently attached to a nucleotide or nucleoside. In other embodiments, the biotin moiety-containing molecule or the analog thereof is a free biotin molecule or an analog that is not covalently attached to a nucleotide or nucleoside. In some embodiments, the terminal nucleotide moiety comprises a 3′ blocking group, and the avidin binding partner comprises a biotin moiety covalently attached to the nucleobase of the terminal nucleotide moiety, optionally through a cleavable linker, and wherein the avidin is conjugated to the terminal nucleotide moiety through non-covalent interaction between the biotin moiety and a biotin-binding site of the avidin. In further embodiments, the oligonucleotide or polynucleotide conjugate is at least partially complementary and hybridized to a target polynucleotide immobilized on a surface of a solid support. In some embodiments of the avidin described herein, avidin may be streptavidin.

Some aspect of the present disclosure relates to a method for preparing a labeled avidin for polynucleotide sequencing as described herein, comprising contacting a biotin moiety-containing molecule or an analog thereof with an avidin labeled with one or more detectable labels (e.g., fluorescent dye moieties). In some embodiments, the molar ratio of biotin moiety-containing molecule or the analog to avidin is about 1:1, 2:1, or 3:1. In some embodiments of the avidin described herein, avidin may be streptavidin. In any embodiments, the biotin moiety-containing molecule or the analog thereof is a free biotin molecule or an analog that is not attached to another compound. In other embodiments, the biotin moiety-containing molecule or the analog thereof is covalently attached to a nucleotide or nucleoside, optionally via a linker, for example, a cleavable linker.

Another aspect of the present disclosure relates to a method for determining the sequences of a plurality of different target polynucleotides in parallel, comprising:
(i) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(ii) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein a first type of nucleotide comprises a first label, a second type of nucleotide comprises a second label, a third type of nucleotide is unlabeled and functionalized with an avidin binding partner, the fourth type of nucleotide is not labeled, and each of the four types of nucleotides comprises a 3′ blocking group;
(iii) imaging and performing a first fluorescent measurement of the extended copy polynucleotides;
(iv) contacting the extended copy polynucleotides with a labeled avidin described herein; and
(v) imaging and performing a second fluorescent measurement of the extended copy polynucleotides.

In some embodiments of the sequencing method described herein, the avidin is streptavidin and the avidin binding partner comprises a biotin moiety covalently attached to the nucleobase of the third type of nucleotide, optionally through a cleavable linker. In some embodiments, the method further comprises contacting the extended copy polynucleotides with a chemical reagent to remove the first label of the first type of nucleotide after the first fluorescent measurement and prior to the second fluorescent measurement. In further embodiments, the first label is covalently attached to the nucleobase of the first type of nucleotide via a cleavable linker comprising an azido, allyl, or disulfide moiety. In some embodiments, the chemical reagent for removing the first label of the first type of nucleotide comprises tris(hydroxypropyl)phosphine (THP).

In some embodiments of the sequencing method described herein, the incorporation of the first type of nucleotide is determined by a signal state in the first fluorescent measurement and a dark state in the second fluorescent measurement. In some embodiments, the incorporation of the second type of nucleotide is determined by a signal state in the first fluorescent measurement and a signal state in the second fluorescent measurement. In some embodiments, the incorporation of the third type of nucleotide is determined by a dark state in the first fluorescent measurement and a signal state in the second fluorescent measurement. In some embodiments, the incorporation of the fourth type of nucleotide is determined by a dark state in the first fluorescent measurement and a dark state in the second fluorescent measurement.

In some embodiments of the sequencing method described herein, the method further comprises (vi) removing the 3′ blocking group of the incorporated nucleotide. In some embodiments, steps (ii) through (vi) are repeated at least 50, 100, 150, 200, 250, 300, 350, 400, 450 or 500 cycles. In some embodiments, the four types of nucleotides comprise dATP, dCTP, dGTP and dTTP or dUTP, or non-natural nucleotide analogs thereof. In further embodiments, the first label, the second label, and the one or more fluorescent moieties of the avidin are identical. In other embodiments, the first label, the second label, and the one or more fluorescent moieties of the avidin in the composition are different by can be excited by the same light source and emits a signal that can be collected in the same detection channel.

A further aspect of the present disclosure relates to another method for determining the sequences of a plurality of different target polynucleotides in parallel, comprising:
(i) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(ii) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein one type of nucleotides is the labeled avidin nucleotide conjugate described herein, and each of the four types of nucleotides comprises a 3′ blocking group;
(iii) imaging and performing one or more fluorescent measurements of the extended copy polynucleotides; and
(iv) removing the 3′ blocking group of the incorporated nucleotides.

In some embodiments of the sequencing method described herein, the aqueous solution of step (ii) comprises a first type of labeled avidin nucleotide conjugate, a second type of labeled nucleotide, a third type of labeled nucleotide, and a fourth type of unlabeled nucleotide. In some embodiments, the aqueous solution of step (ii) comprises a first type of labeled avidin nucleotide conjugate, a second type of labeled nucleotide, a third type of labeled nucleotide, and a fourth type of labeled nucleotide, and the four types of labeled nucleotides are spectrally distinguishable. In some embodiments, steps (ii) through (iv) are repeated at least 50, 100, 150, 200, 250 or 300 cycles. In some embodiments, the four types of nucleotides comprise dATP, dCTP, dGTP and dTTP or dUTP, or non-natural nucleotide analogs thereof.

A further aspect of the present disclosure is a kit for polynucleotide sequencing, comprising: a labeled avidin described herein. In some embodiments, the kit further includes an incorporation composition, comprising a first type of nucleotide having a first label, a second type of nucleotide having a second label, a third type of nucleotide is unlabeled and functionalized with an avidin binding partner, the fourth type of nucleotide is not labeled, and each of the four types of nucleotides comprises a 3′ blocking group. In some embodiments, the avidin is streptavidin and the avidin binding partner comprises a biotin moiety covalently attached to the nucleobase of the third type of nucleotide, optionally through a cleavable linker. In some embodiments, the first label is covalently attached to the nucleobase of the first type of nucleotide via a cleavable linker comprising an azido, allyl, or disulfide moiety. In some embodiments, the kit further comprises a cleavage composition comprising a chemical reagent capable of removing the first label of the first type of nucleotide. In further embodiments, the chemical reagent for removing the first label of the first type of nucleotide comprises tris(hydroxypropyl)phosphine (THP). In some embodiments, the four types of nucleotides comprise dATP, dCTP, dGTP and dTTP or dUTP, or non-natural nucleotide analogs thereof.

Another aspect of the present disclosure relates to a kit for polynucleotide sequencing, comprising a labeled avidin nucleotide conjugate (i.e., a first type of nucleotide having a first label). In further embodiments, the labeled avidin nucleotide conjugate is in an incorporation mix, which further comprises a second type of nucleotide having a second label, a third type of nucleotide that having a third label or having a mixture of the first label and the second label, the fourth type of nucleotide is not labeled or having a fourth label, and each of the four types of nucleotides comprises a 3′ blocking group.

### BRIEF DESCRIPTION OF THE DRAWINGS

FIG. 1A illustrates a flowchart for the standard Illumina iSeq™ one-channel sequencing-by-synthesis (SBS) chemistry.

FIG. 1B illustrates an embodiment of step 3 of FIG. 1A, where a composition containing a cleavage reagent remove the dye from A nucleotide and conjugates multi-dye labeled streptavidin to C nucleotide.

FIG. 2A illustrates the comparative relative fluorescent signal intensity between one fluorescent dye molecule in solution and a multi-dye labeled streptavidin molecule.

FIG. 2B is a scatterplot generated from a sequencing run on Illumina's MiSeq™ system using a six-dye labeled streptavidin conjugated with a biotin-containing ffC.

FIGS. 3A-3C illustrate three possible scenarios in which a biotin moiety on the incorporated nucleotide moiety may interact with the multi-dye labeled streptavidin during one-channel SBS.

FIG. 4 illustrates the variation of blue signals during sequencing on Illumina's MiSeq™ system, when streptavidin is preincubated with various molar ratio of a biotin moiety-containing molecule.

### DETAILED DESCRIPTION

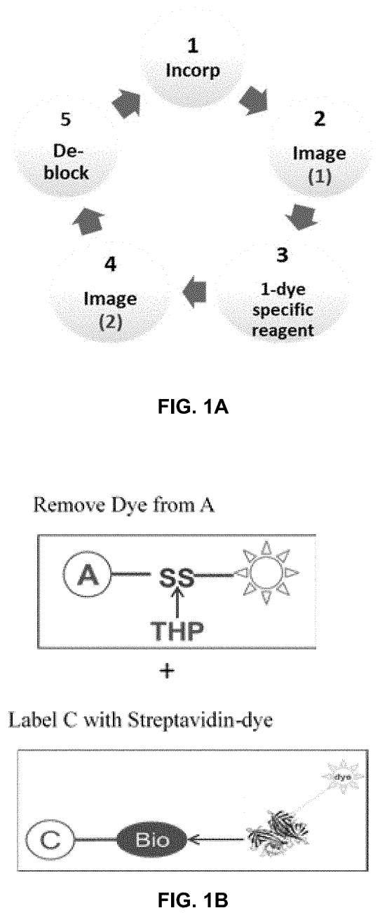

Illumina's Next-Generation Sequencing system, the iSeq™ 100, uses a complementary metal-oxide-semiconductor (CMOS)-based technology to deliver a simplified, accessible benchtop sequencing solution. The standard sequencing workflow is illustrated in FIG. 1A, which is also referred to as the one-channel sequencing-by-synthesis (SBS) chemistry. In contrast to traditional four-channel or two-channel SBS, one-channel SBS requires the cleavage of a di-sulfide bond and conjugation of a nucleobase, covalently attached to a biotin moiety, with fluorescently labeled streptavidin. However, fluorescent quenching during sample preparation and sequencing necessitates alternative methods to enhance optical detection of the fluorescently labeled streptavidin, particularly when the imaging event uses a laser with a shorter wavelength (e.g., a blue laser with wavelength between 450 nm to 460 nm).

Each sequencing cycle in the one-channel SBS includes two chemistry steps and two imaging steps. In FIG. 1A, the first chemistry step exposes the flowcell to a mixture of nucleotides that have fluorescently labeled A and T. C nucleotide is unlabeled and functionalized with a biotin moiety. During the first imaging step, the light emission from each cluster is recorded by the CMOS sensor. The second chemistry step removes the fluorescent label from A and adds a fluorescent label to C. In both chemistry steps, G is dark (unlabeled). The second image is recorded. The combination of Image 1 and Image 2 are processed by image analysis software to identify which bases are incorporated at each cluster position. This sequencing cycle is repeated “n” times to create a read length of “n” bases. Unlike four-channel SBS chemistry, where sequencers use a different dye for each nucleotide, the iSeq™ System uses one dye per sequencing cycle. In FIG. 1B, A has a cleavable disulfide linker that attaches the fluorescent label to the nucleobase, and the label is removed after the first imaging step. C has a biotin moiety that can bind to a labeled streptavidin by non-covalent interaction. Therefore, C is not labeled in the first imaging step but labeled in the second imaging step. T has a permanent fluorescent label and is therefore labeled in both the first and the second imaging steps, and G is permanently dark. Nucleotides are identified by analysis of the different emission patterns for each base across the two images.

The dye labeled streptavidin in FIG. 1B is usually randomly labeled with fluorescent dye using the amino group of lysine on the protein surface to form amide bond with carboxylic acid group on dye. On average, there are six dyes per streptavidin. However, it has been observed that dye labeled streptavidin only gave half of the fluorescent signal comparing to the dye itself in solution. Without being bound by a particular theory, it is suspected dye-dye quenching and dye-protein quenching may be the cause of the loss of fluorescent signal when the dye is attached to streptavidin.

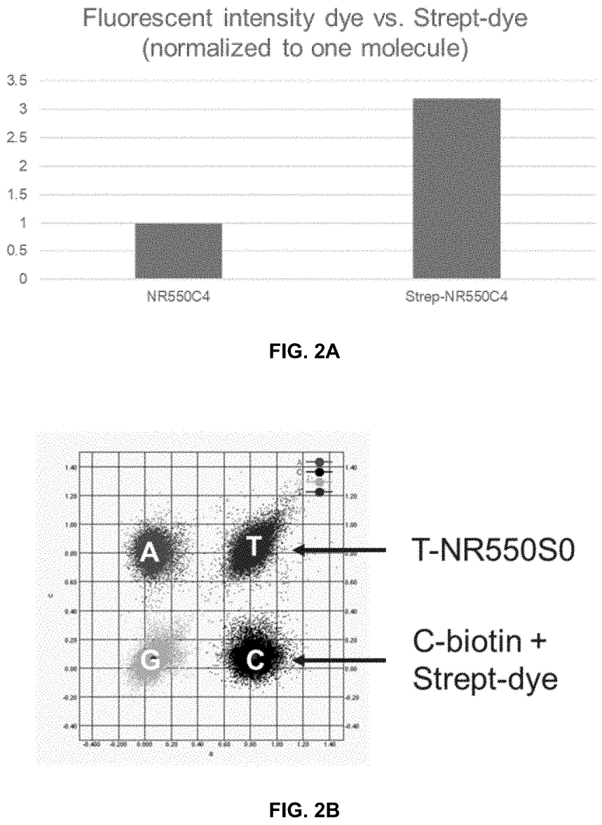

FIG. 2A illustrates the relative fluorescent intensity of a known green dye NR550C4 in solution, as compared to the fluorescent intensity of a multi-dye labeled streptavidin (normalized to one molecule) in solution. The solution data shows that the green dye on streptavidin only gave half of the signal comparing to the dye by itself. Further signal reduction was observed during SBS on the one-channel system. As shown in FIG. 2B, after the multi-dye labeled streptavidin is conjugated with biotin modified C on clusters, it gave only equivalent of same signal as T labeled with a single dye molecule. Therefore, six dyes on streptavidin only resulted to one dye signal intensity on the flowcell.

The present disclosure provides an improved quantitative labeled avidin (e.g., streptavidin) to enhance fluorescent signals on the flowcell during sequencing cycles. In particular, the present disclosure provides labeled avidin/streptavidin where at least one biotin-binding site of the streptavidin is blocked. Blocking up to three biotin-binding sites of the streptavidin may prevent undesirable the same protein from cross-linking with other available biotin molecules on different polynucleotide strands, and improve fluorescent signals during sequencing.

### Definitions

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
LED Light emitting diode
SBS Sequencing by synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the term “non-covalent interactions” differs from a covalent bond in that it does not involve the sharing of electrons, but rather involves more dispersed variations of electromagnetic interactions between molecules or within a molecule. Non-covalent interactions can be generally classified into four categories, electrostatic, π-effects, van der Waals forces, and hydrophobic effects. Non-limiting examples of electrostatic interactions include ionic interactions, hydrogen bonding (a specific type of dipole-dipole interaction), halogen bonding, etc. Van der Walls forces are a subset of electrostatic interaction involving permanent or induced dipoles or multipoles. π-effects can be broken down into numerous categories, including (but not limited to) π-π interactions, cation-π & anion-π interactions, and polar-π interactions. In general, π-effects are associated with the interactions of molecules with the π-orbitals of a molecular system, such as benzene. The hydrophobic effect is the tendency of nonpolar substances to aggregate in aqueous solution and exclude water molecules. Non-covalent interactions can be both intermolecular and intramolecular. Non-covalent interactions can be both intermolecular and intramolecular.

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e., a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleotide conjugate” generally refers to a nucleotide labeled with a fluorescent moiety, optionally through a cleavage linker as described herein. In some embodiment, when a nucleotide conjugate is described as an unlabeled nucleotide, such nucleotide does not include a fluorescent moiety. In some further embodiments, an unlabeled nucleotide conjugate also does not have a cleavable linker.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative,” “analog” and “modified” as used herein, may be used interchangeably, and are encompassed by the terms “nucleotide” and “nucleoside” defined herein.

As used herein, a biotin moiety-containing molecule or an analog thereof comprise the biotin moiety of structure

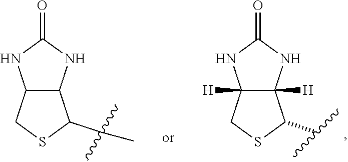 [SMILES: *C1SCC2NC(=O)NC21 ; *[C@@H]1SC[C@@H]2NC(=O)N[C@@H]21]

or more specifically

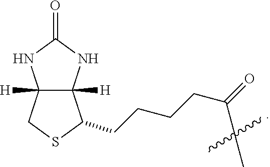 [SMILES: *C(=O)CCCC[C@@H]1SC[C@@H]2NC(=O)N[C@@H]21]

The analog of the biotin moiety-containing molecule may include a substituted biotin moiety.

As used herein, the term “phosphate” is used in its ordinary sense as understood by those skilled in the art, and includes its protonated forms (for example,

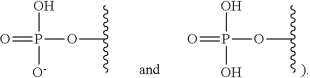 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

As understood by one of ordinary skill in the art, a compound such as a nucleotide conjugate described herein may exist in ionized form, e.g., containing a —CO₂⁻, —SO₃⁻ or —O⁻. If a compound contains a positively or negatively charged substituent group, it may also contain a negatively or positively charged counterion such that the compound as a whole is neutral. In other aspects, the compound may exist in a salt form, where the counterion is provided by a conjugate acid or base.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Prephasing is caused by the incorporation of nucleotides without effective 3′ terminators, wherein the incorporation event goes 1 cycle ahead due to a termination failure. Phasing and prephasing cause the measured signal intensities for a specific cycle to consist of the signal from the current cycle as well as noise from the preceding and following cycles. As the number of cycles increases, the fraction of sequences per cluster affected by phasing and prephasing increases, hampering the identification of the correct base. Prephasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes.

### Labeled Avidin

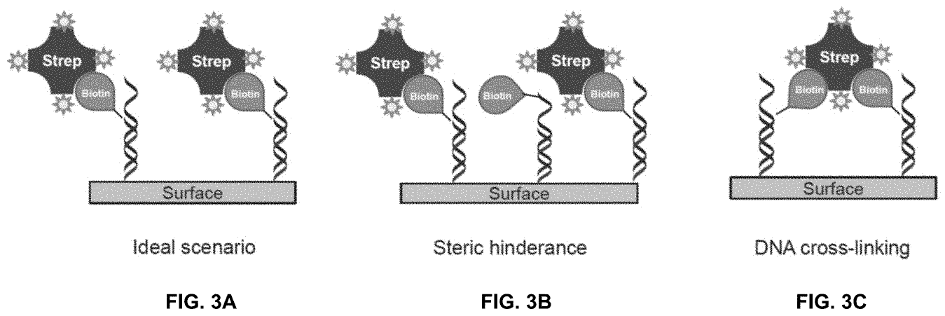

As described herein, a multi-dye labeled streptavidin used in one-channel SBS results in substantial fluorescent signal loss during sequencing. FIG. 3A illustrates the ideal scenario where each copy polynucleotide strand incorporating the biotin modified nucleotide is conjugated to one molecule of streptavidin labeled with multiple dyes. FIG. 3B illustrates one possible scenario of signal loss during sequencing that is caused by steric hinderance of streptavidin. Because the flowcell has densely packed clusters, one polynucleotide strand might not be able to conjugate to labeled streptavidin when an adjacent polynucleotide strand is conjugated to streptavidin. FIG. 3C illustrates another possible scenario of signal loss due to DNA cross-linking. In this case, the multi-dye labeled streptavidin has more than one biotin-binding sites available so two biotin modified nucleotides incorporated by two different copy polynucleotide strands are conjugated to the same streptavidin. The present application solves the problem of sequencing signal loss caused by DNA cross-linking.

Some aspect of the present disclosure relates to a labeled avidin for polynucleotide sequencing, wherein the avidin molecule is labeled with one or more fluorescent dye moieties, wherein at least one biotin-binding site of the avidin is blocked with a biotin moiety-containing molecule or an analog thereof, and wherein the labeled avidin comprises at least one open biotin-binding site.

In some embodiments of the labeled avidin/streptavidin described herein, one to three biotin-binding sites of the streptavidin are blocked with the biotin moiety-containing molecule or the analog thereof. In some embodiments, one biotin-binding site of the streptavidin is blocked with the biotin moiety-containing molecule or the analog thereof. In some embodiments, two biotin-binding sites of the streptavidin are blocked with the biotin moiety-containing molecule or the analog thereof. In some embodiments, three biotin-binding sites of the streptavidin are blocked with the biotin moiety-containing molecule or the analog thereof. In some embodiments, the biotin moiety-containing molecule or the analog thereof is a free biotin molecule or an analog that is not covalently attached to a nucleotide or nucleoside. For example, the biotin moiety-containing molecule or the analog thereof may comprise a free biotin

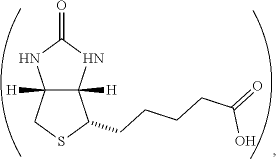

biotin-TEG, dual biotin, PC biotin, desthiobiotin-TEG, biotin azide, or combinations thereof. In other embodiments, the biotin moiety-containing molecule or the analog thereof comprises a biotin moiety that is covalently attached to a nucleotide or nucleoside, optionally via a linker (e.g., a cleavable linker). In some embodiments of the labeled avidin/streptavidin described herein, avidin/streptavidin is labeled with two to eight fluorescent dye moieties. In some embodiments, avidin/streptavidin is labeled with two fluorescent dye moieties. In some embodiments, avidin/streptavidin is labeled with three fluorescent dye moieties. In some embodiments, avidin/streptavidin is labeled with four fluorescent dye moieties. In some embodiments, avidin/streptavidin is labeled with five fluorescent dye moieties. In some embodiments, avidin/streptavidin is labeled with six fluorescent dye moieties.

In some embodiments of the labeled avidin/streptavidin described herein, the fluorescent dye moieties are identical and/or spectrally indistinguishable. In some embodiments, the fluorescent dye moieties of the avidin/streptavidin are excitable by a light source have a wavelength between about 400 nm to about 650 nm. For example, the light source may have a wavelength of about 400 nm, 410 nm, 420 nm, 430 nm, 440 nm, 445 nm, 450 nm, 455 nm, 460 nm, 465 nm, 470 nm, 480 nm, 490 nm, 500 nm, 510 nm, 515 nm, 520 nm, 525 nm, 530 nm, 535 nm, 540 nm, 545 nm, 550 nm, 560 nm, 570 nm, 580 nm, 590 nm, 600 nm, 610 nm, 620 nm, 630 nm, 640 nm or 650 nm, or a range defined by any two of the preceding values. For example, a wavelength in any one of the following ranges: about 440-470 nm, about 450-460 nm, about 510-545 nm, 515-540 nm, 520-535 nm, or 525-535 nm. In some further embodiments, the fluorescent dye moieties are excitable by a light source having a wavelength between about 450 nm to about 460 nm, or between about 520 nm to about 535 nm.

In any embodiments of the labeled avidin described herein, the avidin is streptavidin. In other embodiments, the avidin may be neutravidin.

In some embodiments of the labeled avidin/streptavidin described herein, the labeled avidin/streptavidin is in an aqueous composition. In further embodiments, the composition may comprise additional chemical reagent(s), for example, a cleavage reagent for removing a label from a labeled nucleotide, which is described in detail below.

### Avidin Nucleotide/Oligonucleotide/Polynucleotide Conjugates

One aspect of the present disclosure relates to a labeled avidin nucleotide conjugate, comprising an avidin-binding partner covalently attached to the nucleotide, optionally via a cleavable linker, wherein the avidin-binding partner is conjugated with the labeled avidin described herein via non-covalent interaction. In some embodiments, the avidin-binding partner comprise a biotin moiety. In further embodiments, the avidin-binding partner is covalently attached to the nucleobase of the nucleotide via a cleavable linker described herein, for example, the cleavable linker may comprise an azido, allyl, or disulfide moiety. In further embodiments, the nucleotide conjugate comprises a 3′ blocking group. In further embodiments, the nucleotide is nucleotide triphosphate with 2′ deoxyribose.

Another aspect of the present disclosure relates to an oligonucleotide or polynucleotide conjugate, comprising an avidin conjugated to a terminal nucleotide moiety of the oligonucleotide or polynucleotide, wherein the avidin is labeled with one or more fluorescent dye moieties, and at least one biotin-binding sites of the avidin is blocked with a biotin moiety-containing molecule or an analog thereof, wherein the terminal nucleotide moiety comprises an avidin binding partner covalently attached thereto, and the labeled avidin is conjugated to the avidin binding partner of the terminal nucleotide moiety by non-covalent interaction. In some embodiments, the oligonucleotide/polynucleotide comprises the labeled avidin nucleotide conjugate incorporated therein. In some embodiments, the avidin is streptavidin.

In some embodiments of the nucleotide/oligonucleotide/polynucleotide conjugate described herein, one to three biotin-binding sites of the avidin/streptavidin are blocked with the biotin moiety-containing molecule or the analog thereof. In some embodiments, the terminal nucleotide moiety comprises a 3′ blocking group, and the avidin/streptavidin binding partner comprises a biotin moiety covalently attached to the nucleobase of nucleotide or the terminal nucleotide moiety or the oligonucleotide/polynucleotide, optionally through a cleavable linker, and wherein the avidin/streptavidin is conjugated to the nucleotide moiety through non-covalent interaction between the biotin moiety and a biotin-binding site of the avidin/streptavidin. In some embodiments, one, two or three biotin-binding sites of the avidin/streptavidin is blocked with the biotin moiety-containing molecule or the analog thereof. In some embodiments, the biotin moiety-containing molecule or the analog thereof comprises a biotin moiety covalently attached to a nucleotide or nucleoside, optionally via a linker. In other embodiments, the biotin moiety-containing molecule or the analog thereof is a free biotin molecule or an analog that is not covalently attached to a nucleotide or nucleoside. For example, the biotin moiety-containing molecule or the analog thereof may comprise a free biotin, biotin-TEG, dual biotin, PC biotin, desthiobiotin-TEG, biotin azide, or combinations thereof. In further embodiments, the oligonucleotide or polynucleotide conjugate is at least partially complementary and hybridized to a target polynucleotide immobilized on a surface of a solid support. In further embodiments, the solid support contains an array of a plurality of immobilized target polynucleotides. In one embodiment, the solid support comprises or is a flowcell.

In any embodiments of the oligonucleotide/polynucleotide conjugate described herein, the avidin is streptavidin. In other embodiments, the avidin may be neutravidin.

### Method for Preparing Labeled Avidin

Some aspect of the present disclosure relates to a method for preparing the labeled avidin disclosed herein, comprising contacting a biotin moiety-containing molecule or an analog thereof with avidin labeled with one or more fluorescent dye moieties. In some embodiment, the method blocks one or more biotin-binding sites of the avidin. In some embodiments, the avidin is streptavidin. In some embodiments, the molar ratio of the biotin moiety-containing molecule or the analog to avidin/streptavidin is about 1:1. In some embodiments, the molar ratio of biotin moiety-containing molecule or the analog to avidin/streptavidin is about 2:1. In some embodiments, the molar ratio of biotin moiety-containing molecule or the analog to avidin/streptavidin is about 3:1. In some embodiments, wherein the biotin moiety-containing molecule or the analog thereof is a free biotin molecule or an analog that is not covalently attached to a nucleotide or nucleoside. For example, the biotin moiety-containing molecule or the analog thereof that blocking the biotin-binding sites of the avidin/streptavidin may comprise a nucleotide or nucleoside with a covalently attached biotin moiety, optionally via a linker free biotin, biotin-TEG, dual biotin, PC biotin, desthiobiotin-TEG, biotin azide, or combinations thereof.

### Fluorescent Dyes

Various fluorescent dyes may be used in the present disclosure as detectable labels, in particularly those dyes that may be excitation by a blue light or a green light. These dyes may also be referred to as “blue dyes” and “green dyes” respectively. Examples of various type of blue dyes, including but not limited to coumarin dyes, chromenoquinoline dyes, and bisboron containing heterocycles are disclosed in U.S. Publication Nos. 2018/0094140, 2018/0201981, 2020/0277529, 2020/0277670, 2021/0188832, 2022/0033900, 2022/0195517 A1, 2022/0380389 A1 and U.S. Ser. No. 63/325,057, each of which is incorporated by reference in its entirety. Non-limiting examples of the blue dyes include:

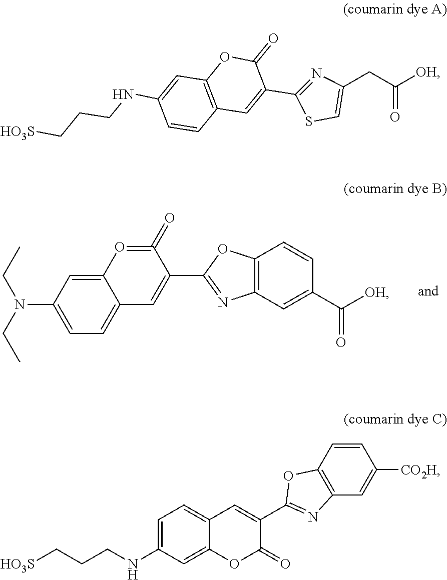

and salts, mesomeric forms, and optionally substituted analogs thereof. For example, analogs with —SO₃H substitution on the alkyl group(s).

Examples of green dyes including cyanine or polymethine dyes disclosed in International Publication Nos. WO2013/041117, WO2014/135221, WO2016/189287, WO2017/051201 and WO2018/060482A1, each of which is incorporated by reference in its entirety. Non-limiting examples of the green dyes include:

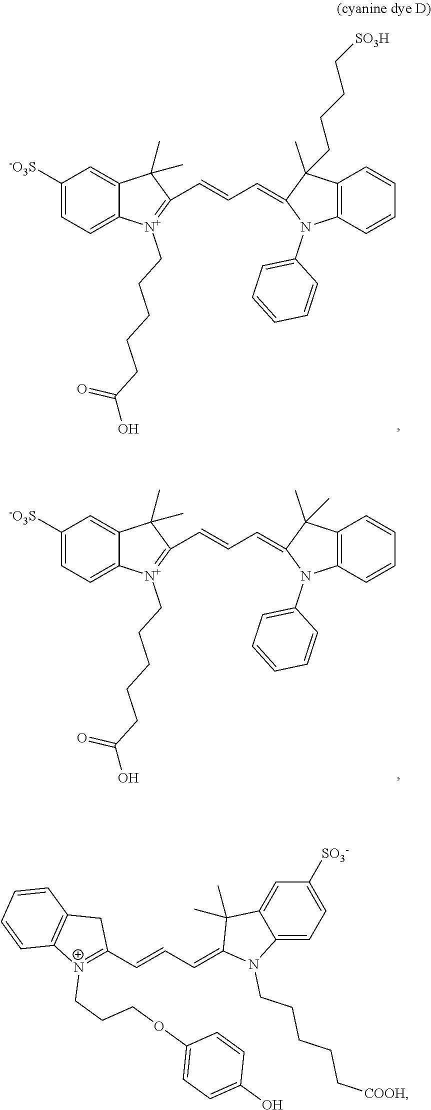

and salts, mesomeric forms, and optionally substituted analogs thereof.

In some embodiments, the fluorescent dyes described herein may be further modified to introduce one or more substituents (such as —SO₃H, —OH, —C(O)OH, —C(O)OR, where R is unsubstituted or substituted C₁-C₆ alkyl) to improve the hydrophilicity of the dyes when the dyes are conjugated with the avidin described herein, while maintaining the signal intensity of the dye.

### Kits

A further aspect of the present disclosure is a kit for polynucleotide or nucleic acid sequencing, comprising:
an incorporation composition comprising a first type of nucleotide having a first label, a second type of nucleotide having a second label, a third type of nucleotide is unlabeled and functionalized with an avidin binding partner, the fourth type of nucleotide is not labeled, and each of the four types of nucleotides comprises a 3′ hydroxy blocking group; and
a labeled avidin described herein, where at least one biotin-binding sites of the avidin is blocked with a biotin moiety-containing molecule or an analog thereof. In some embodiments, the kit is used in one-channel SBS described in FIG. 1A.

In some embodiments of the kits described herein, the avidin is streptavidin and the avidin binding partner comprises a biotin moiety covalently attached to the nucleobase of the third type of nucleotide, optionally through a cleavable linker. In some embodiments, the first label is covalently attached to the nucleobase of the first type of nucleotide via a cleavable linker comprising an azido, allyl, or disulfide moiety. In some embodiments, the kit further comprises a cleavage composition comprising a chemical reagent capable of removing the first label of the first type of nucleotide. In further embodiments, the chemical reagent for removing the first label of the first type of nucleotide comprises tris(hydroxypropyl)phosphine (THP). In some embodiments, the four types of nucleotides comprise dATP, dCTP, dGTP and dTTP or dUTP, or non-natural nucleotide analogs thereof.

Another aspect of the present disclosure relates to a kit for polynucleotide sequencing, comprising a labeled avidin nucleotide conjugate (a first type of nucleotide having a first label) described herein. In some embodiments, the kit is for two-channel SBS or four-channel SBS. In further embodiments, the labeled avidin nucleotide conjugate is in an incorporation mix. For two-channel sequencing where two light sources with different wavelengths are used, the incorporation mix may further comprise a second type of nucleotide having a second label, a third type of nucleotide having both the first label and the second label, and the fourth type of nucleotide that is not labeled, and each of the four types of nucleotides comprises a 3′ blocking group. For four-channel sequencing where four light sources with different wavelengths are used, the incorporation mix may further comprise a second type of nucleotide having a second label, a third type of nucleotide having a third label, and the fourth type of nucleotide having a fourth label, wherein the first, second, third and fourth labels are spectrally distinguishable, and each of the four types of nucleotides comprises a 3′ blocking group.

In some embodiments of the kits described herein, the labeled avidin/streptavidin are excitable by a light source have a wavelength between about 400 nm to about 650 nm. For example, the light source may have a wavelength of about 400 nm, 410 nm, 420 nm, 430 nm, 440 nm, 445 nm, 450 nm, 455 nm, 460 nm, 465 nm, 470 nm, 480 nm, 490 nm, 500 nm, 510 nm, 515 nm, 520 nm, 525 nm, 530 nm, 535 nm, 540 nm, 545 nm, 550 nm, 560 nm, 570 nm, 580 nm, 590 nm, 600 nm, 610 nm, 620 nm, 630 nm, 640 nm or 650 nm, or a range defined by any two of the preceding values. For example, a wavelength in any one of the following ranges: 440-470 nm, 450-460 nm, 510-545 nm, 515-540 nm, 520-535 nm, or 525-535 nm. In some further embodiments, the fluorescent dye moieties are excitable by a light source having a wavelength between about 450 nm to about 460 nm, or between about 520 nm to about 535 nm.

In a particular embodiment, the labeled nucleotide(s) described herein may be supplied in combination with unlabeled or native nucleotides, or any combination thereof. Combinations of nucleotides may be provided as separate individual components (e.g., one nucleotide type per vessel or tube) or as nucleotide mixtures (e.g., two or more nucleotides mixed in the same vessel or tube).

Where kits comprise a plurality, particularly two, or three, or more particularly four, nucleotides, the different nucleotides may be labeled with different dye compounds, or one may be dark, with no dye compounds. Where the different nucleotides are labeled with different dye compounds, it is a feature of the kits that the dye compounds are spectrally distinguishable fluorescent dyes. As used herein, the term “spectrally distinguishable fluorescent dyes” refers to fluorescent dyes that emit fluorescent energy at wavelengths that can be distinguished by fluorescent detection equipment (for example, a commercial capillary-based DNA sequencing platform) when two or more such dyes are present in one sample.

Although kits are exemplified herein in regard to configurations having different nucleotides that are labeled with different dye compounds, it will be understood that kits can include 2, 3, 4 or more different nucleotides that have the same dye compound or different dye compounds that may be excited by the same light source.

In addition to the labeled nucleotides, the kit may comprise together at least one additional component. The further component(s) may be one or more of the components identified in a method set forth herein or in the Examples section below. Some non-limiting examples of components that can be combined into a kit of the present disclosure are set forth below. In some embodiments, the kit further comprises a DNA polymerase (such as a mutant DNA polymerase) and one or more buffer compositions. Non-limiting examples of DNA polymerase may be used in the present disclosure include those disclosed in WO 2005/024010, US Publication Nos. 2020/0131484 A1 and 2020/0181587 A1, each of which is incorporated by reference herein in its entirety. One buffer composition may comprise antioxidants such as ascorbic acid, sodium ascorbate, gallic acid or a salt or ester thereof, which can be used to protect the dye compounds from photo damage during detection. Additional buffer composition may comprise a reagent can may be used to cleave the 3′ blocking group and/or the cleavable linker. For example, a water-soluble phosphines or water-soluble transition metal catalysts formed from a transition metal and at least partially water-soluble ligands, such as a palladium complex. Various components of the kit may be provided in a concentrated form to be diluted prior to use. In such embodiments a suitable dilution buffer may also be included. Again, one or more of the components identified in a method set forth herein can be included in a kit of the present disclosure. In any embodiments of the nucleotide or labeled nucleotide described herein, the nucleotide contains a 3′ blocking group.

3′ Blocking Groups

The labeled nucleotide described herein may also have a 3′ blocking group covalently attached to the deoxyribose sugar of the nucleotide. Various 3′ blocking group are disclosed in WO2004/018497 and WO2014/139596, which are hereby incorporated by references. For example, the blocking group may be azidomethyl (—CH₂N₃) or substituted azidomethyl (e.g., —CH(CHF₂)N₃ or CH(CH₂F)N₃), or allyl, each connecting to the 3′ oxygen atom of the deoxyribose moiety. In some embodiments, the 3′ blocking group is azidomethyl, forming 3′-OCH₂N₃ with the 3′ carbon of the ribose or deoxyribose.

Additional 3′ blocking groups are disclosed in U.S. Publication No. 2020/0216891 A1, which is incorporated by reference in its entirety. Non-limiting examples of the acetal blocking group

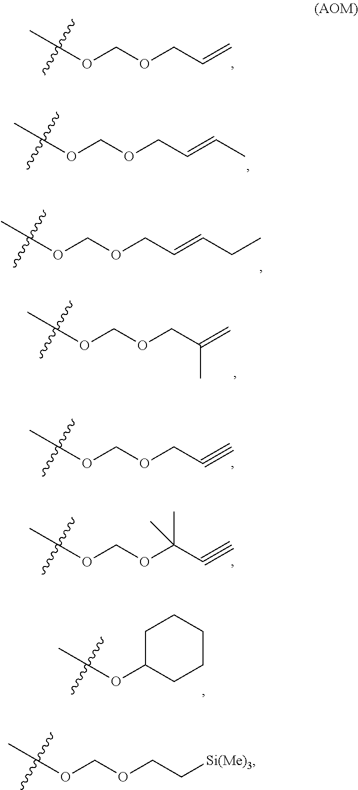

each covalently attached to the 3′ carbon of the deoxyribose.

Deprotection of the 3′ Blocking Groups

In some embodiments, the 3′ hydroxy protecting group such as azidomethyl may be removed or deprotected by using a water soluble phosphine reagent to generate a free 3′-OH. Non-limiting examples include tris(hydroxymethyl)phosphine (THMP), tris(hydroxyethyl)phosphine (THEP) or tris(hydroxypropyl)phosphine (THP or THPP). 3′-acetal blocking groups described herein may be removed or cleaved under various chemical conditions. For acetal blocking groups that contain a allyl moiety, non-limiting cleaving condition includes a Pd(II) complex, such as Pd(OAc)₂ or allylPd(II) chloride dimer, in the presence of a phosphine ligand, for example tris(hydroxymethyl)phosphine (THMP), or tris(hydroxypropyl)phosphine (THP or THPP). For those blocking groups containing an alkynyl group (e.g., an ethynyl), they may also be removed by a Pd(II) complex (e.g., Pd(OAc)₂ or allyl Pd(II) chloride dimer) in the presence of a phosphine ligand (e.g., THP or THMP).

Palladium Cleavage Reagents

In some other embodiments, the 3′ blocking group described herein such as allyl or AOM may be cleaved by a palladium catalyst. In some such embodiments, the Pd catalyst is water soluble. In some such embodiments, is a Pd(0) complex (e.g., Tris(3,3′,3″-phosphinidynetris(benzenesulfonato)palladium(0) nonasodium salt nonahydrate). In some instances, the Pd(0) complex may be generated in situ from reduction of a Pd(II) complex by reagents such as alkenes, alcohols, amines, phosphines, or metal hydrides. Suitable palladium sources include Na₂PdCl₄, Li₂PdCl₄, Pd(CH₃CN)₂Cl₂, (PdCl(C₃H₅))₂, [Pd(C₃H₅)(THP)]Cl, [Pd(C₃H₅)(THP)₂]Cl, Pd(OAc)₂, Pd(Ph₃)₄, Pd(dba)₂, Pd(Acac)₂, PdCl₂(COD), Pd(TFA)₂, Na₂PdBr₄, K₂PdBr₄, PdCl₂, PdBr₂, and Pd(NO₃)₂. In one such embodiment, the Pd(0) complex is generated in situ from Na₂PdCl₄ or K₂PdCl₄. In another embodiment, the palladium source is allyl palladium(II) chloride dimer [(PdCl(C₃H₅))₂]. In some embodiments, the Pd(0) complex is generated in an aqueous solution by mixing a Pd(II) complex with a phosphine. Suitable phosphines include water soluble phosphines, such as THP, THMP, PTA, TCEP, bis(p-sulfonatophenyl)phenylphosphine dihydrate potassium salt, or triphenylphosphine-3,3′,3″-trisulfonic acid trisodium salt.

In some embodiments, the palladium catalyst is prepared by mixing [(Allyl)PdCl]₂ with THP in situ. The molar ratio of [(Allyl)PdCl]₂ and the THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of [(Allyl)PdCl]₂ to THP is 1:10. In some other embodiment, the palladium catalyst is prepared by mixing a water soluble Pd reagent such as Na₂PdCl₄ or K₂PdCl₄ with THP in situ. The molar ratio of Na₂PdCl₄ or K₂PdCl₄ and THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of Na₂PdCl₄ or K₂PdCl₄ to THP is about 1:3. In another embodiment, the molar ratio of Na₂PdCl₄ or K₂PdCl₄ to THP is about 1:3.5. In yet another embodiment, the molar ratio of Na₂PdCl₄ or K₂PdCl₄ to THP is about 1:2.5. In some further embodiments, one or more reducing agents may be added, such as ascorbic acid or a salt thereof (e.g., sodium ascorbate). In some embodiments, the cleavage mixture may contain additional buffer reagents, such as a primary amine, a secondary amine, a tertiary amine, a carbonate salt, a phosphate salt, or a borate salt, or combinations thereof. In some further embodiments, the buffer reagent comprises ethanolamine (EA), tris(hydroxymethyl)aminomethane (Tris), glycine, sodium carbonate, sodium phosphate, sodium borate, 2-dimethylethanolamine (DMEA), 2-diethylethanolamine (DEEA), N,N,N′,N′-tetramethylethylenediamine (TEMED), N,N,N′,N′-tetraethylethylenediamine (TEEDA), or 2-piperidine ethanol (also known as (2-hydroxyethyl)piperidine, having the structure

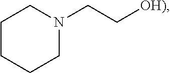

or combinations thereof. In one embodiment, the buffer reagent comprises or is DEEA. In another embodiment, the buffer reagent comprises or is (2-hydroxyethyl)piperidine. In another embodiment, the buffer reagent contains one or more inorganic salts such as a carbonate salt, a phosphate salt, or a borate salt, or combinations thereof. In one embodiment, the inorganic salt is a sodium salt.

Cleavable Linkers

In some embodiments, the avidin-binding partner (such as a biotin moiety) of nucleotide described herein is covalently attached to the nucleobase of the nucleotide via a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

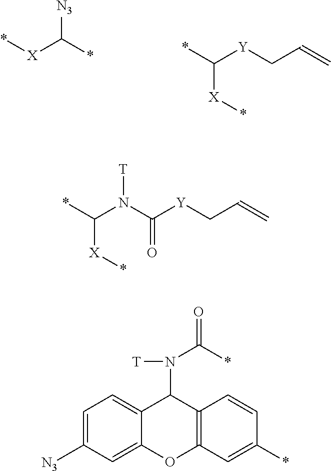

(wherein X is selected from the group comprising O, S, NH and NQ wherein Q is a C₁₋₁₀ substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

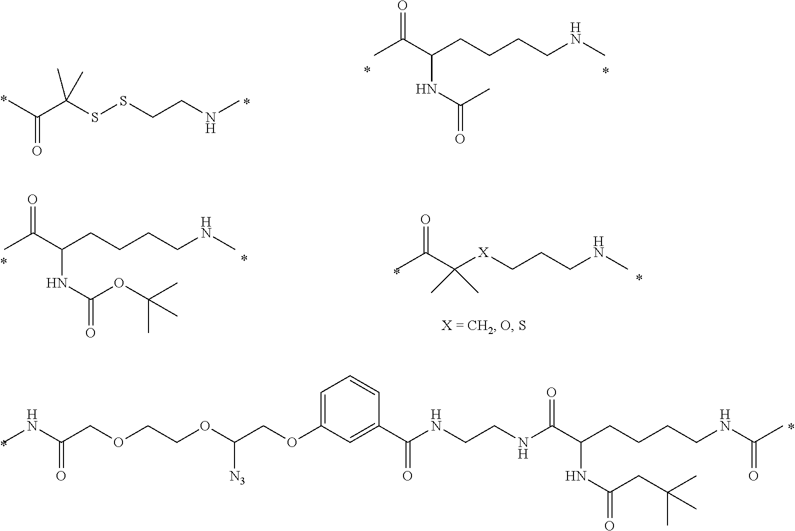

(wherein * indicates where the moiety is connected to the remainder of the nucleotide or nucleoside). The linker moieties illustrated herein may comprise the whole or partial linker structure between the nucleotides/nucleosides and the labels. The linker moieties illustrated herein may comprise the whole or partial linker structure between the nucleotides/nucleosides and the labels.

Additional examples of linkers are disclosed in U.S. Publication No. 2020/0216891 A1, which is incorporated by reference in its entirety:

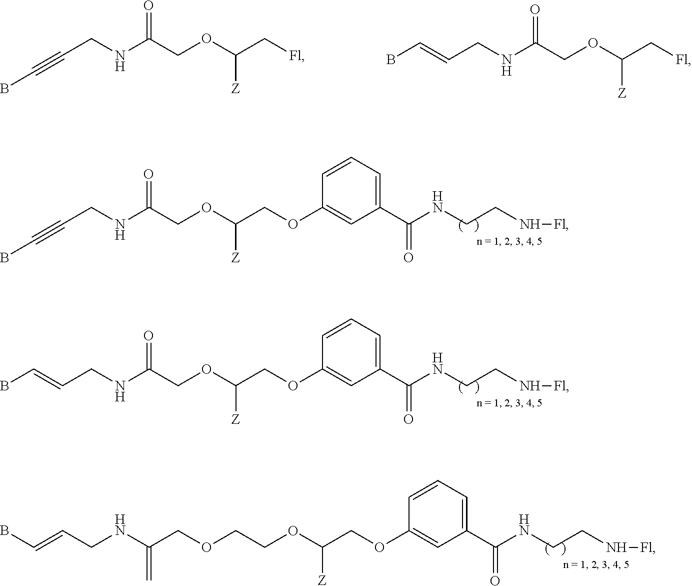

wherein B is a nucleobase; Z is —N₃ (azido), —O—C₁-C₆ alkyl, —O—C₂-C₆ alkenyl, or —O—C₂-C₆ alkynyl; and Fl comprises a dye moiety, which may contain additional linker structure. One of ordinary skill in the art understands that the dye compound described herein is covalently bound to the linker by reacting a functional group of the dye compound (e.g., carboxyl) with a functional group of the linker (e.g., amino). In one embodiment, the cleavable linker comprises

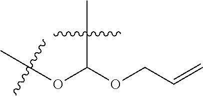 [SMILES: *OC(*)OCC=C]

(“AOL” linker moiety) where Z is —O-allyl.

An avidin binding partner may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleotide.

In particular embodiments the labeled nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit, such as one or more PEG unit(s) (—OCH₂CH₂—)ₙ, where n is an integer of 1-20, for example 1, 2, 3, 4, 5, 6, 7, 8, 9, 10, 11, 12, 13, 14 or 15. The spacer distances, for example, the nucleotide base from a cleavage site or label.

Avidin labeled nucleosides or nucleotides described herein may have the formula:

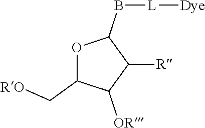

where Dye is multi-dye labeled avidin described herein where one or more biotin binding sites is blocked; B is a nucleobase, such as, for example uracil, thymine, cytosine, adenine, 7-deaza adenine, guanine, 7-deaza guanine, and the like; L is an optional linker which may or may not be present; R′ can be H, a reactive phosphorous containing group, a blocking group, or —OR′ is monophosphate, diphosphate, triphosphate, thiophosphate, or a phosphate ester analog,; R″ is H or OH; and R″′ is H, a 3′ hydroxy blocking group described herein, or —OR″′ forms a phosphoramidite. Where —OR″′ is phosphoramidite, R′ is an acid-cleavable hydroxy protecting group which allows subsequent monomer coupling under automated synthesis conditions. In some further embodiments, B comprises

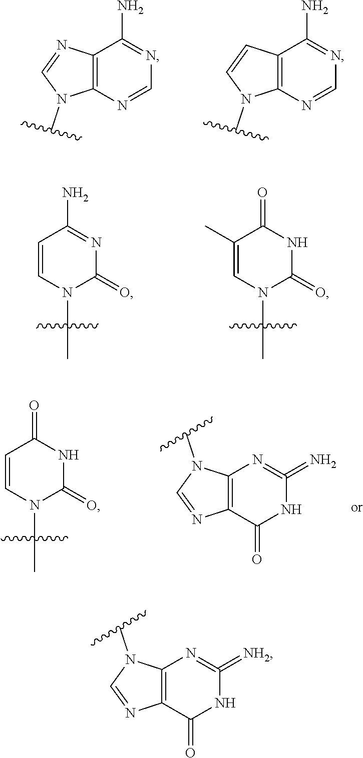

or optionally substituted derivatives and analogs thereof. In some further embodiments, the labeled nucleobase comprises the structure

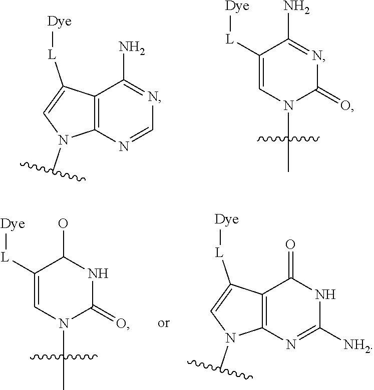

In yet another alternative embodiment, there is no blocking group on the 3′ carbon of the pentose sugar and the labeled avidin attached to the base via a linker, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

The use of a blocking group allows polymerization to be controlled, such as by stopping extension when a labeled nucleotide is incorporated. If the blocking effect is reversible, for example, by way of non-limiting example by changing chemical conditions or by removal of a chemical block, extension can be stopped at certain points and then allowed to continue.

In a particular embodiment, the linker (between labeled avidin and nucleotide) and blocking group are both present and are separate moieties. In particular embodiments, the linker and blocking group are both cleavable under the same or substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

The disclosure also encompasses polynucleotides incorporating dye compounds. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

Non-limiting exemplary labeled nucleotides as described herein include:

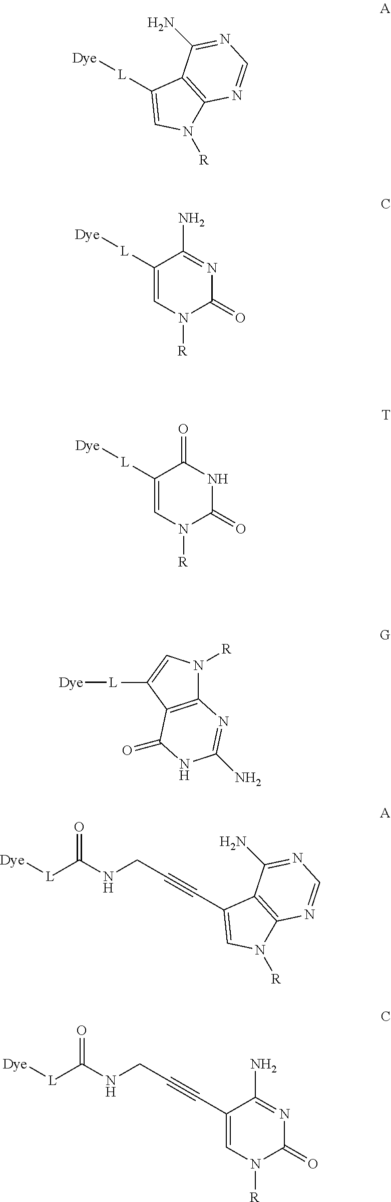

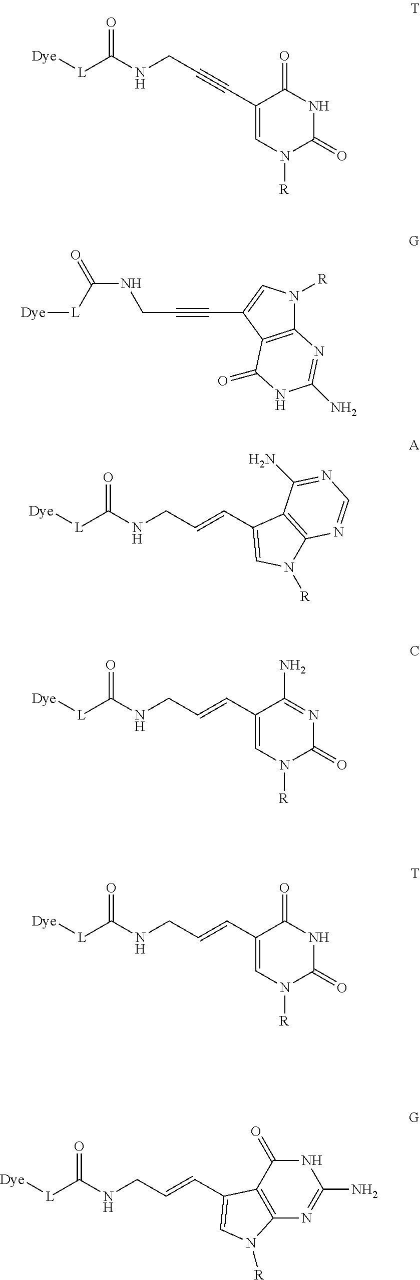

wherein L represents a linker and R represents a ribose or deoxyribose moiety as described above, or a ribose or deoxyribose moiety with the 5′ position substituted with mono-, di- or tri-phosphates.

### Methods of Sequencing

Additional aspect of the present disclosure relates to a method for determining the sequences of a plurality of different target polynucleotides in parallel, comprising:
(i) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(ii) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein a first type of nucleotide comprises a first label, a second type of nucleotide comprises a second label, a third type of nucleotide is unlabeled and functionalized with an avidin binding partner, the fourth type of nucleotide is not labeled, and each of the four types of nucleotides comprises a 3′ hydroxy blocking group;
(iii) imaging and performing a first fluorescent measurement of the extended copy polynucleotides;
(iv) contacting the extended copy polynucleotides with an avidin composition described herein; and
(v) imaging and performing a second fluorescent measurement of the extended copy polynucleotides.

In some embodiments of the sequencing method described herein, the avidin is streptavidin and the avidin binding partner comprises a biotin moiety covalently attached to the nucleobase of the third type of nucleotide, optionally through a cleavable linker.

In some embodiments of the sequencing method described herein, the incorporation of the first type of nucleotide is determined by a signal state in the first fluorescent measurement and a dark state in the second fluorescent measurement. In some embodiments, the incorporation of the second type of nucleotide is determined by a signal state in the first fluorescent measurement and a signal state in the second fluorescent measurement. In some embodiments, the incorporation of the third type of nucleotide is determined by a dark state in the first fluorescent measurement and a signal state in the second fluorescent measurement. In some embodiments, the incorporation of the fourth type of nucleotide is determined by a dark state in the first fluorescent measurement and a dark state in the second fluorescent measurement.

The term “signal state” when used in reference to an imaging event, refers to the state of a polynucleotide incorporating a labeled nucleotide or conjugates to a labeled avidin described herein, in which a specific emission signal is produced by such imaging event, and the emission signal is detected or collected in a single detection channel/filter described herein (i.e., one channel detection). For example, a fluorescent moiety in the incorporated labeled nucleotide may be excited by a light source (e.g., a laser) at a specific wavelength and emits a fluorescent signal that is collected or detected in the single emission detection channel/filter, indicating a “signal state” in such imaging event.

The term “dark state,” when used in reference to an imaging event, refers to the state of to the state of a polynucleotide incorporating a nucleotide, in which no specific emission signal is produced by such imaging event, or no emission signal is collected or detected in the single emission detection channel/filter. A “dark state” of a nucleotide conjugate may result from various situations. In one scenario, such nucleotide conjugate lacks a fluorescent moiety and as a result, it does not emit any fluorescent signals. In another scenario, the nucleotide conjugate is labeled with a fluorescent moiety that cannot be excited by a light source at a specific wavelength and therefore cannot emit any signal or emits minimal fluorescence (e.g., a red emission dye may not be excitable at a wavelength in the blue or violet region). In a third scenario, the nucleotide conjugate is labeled with a fluorescent moiety, and emits a signal as a result of the imaging event. However, the wavelength of such emission signal is outside the single detection channel and thus cannot be detected (e.g., a dye emits a blue signal, but the detection channel is in the green to red region). Dark state detection may also include any background fluorescence which may be present absent a fluorescent label. For example, some reaction components may demonstrate minimal fluorescence when excited at certain wavelengths. As such, even though there is not a fluorescent moiety present there may be background fluorescence from such components. Further, background fluorescence may be due to light scatter, for example from adjacent sequencing reactions, which may be detected by a detector. In addition, background fluorescence may be caused by impure clusters (e.g., due to multiple templates seeding during cluster amplification, phasing or prephasing events). As such, “dark state” can include such background fluorescence as when a fluorescent moiety is not specifically included, such as when a nucleotide lacking a fluorescent label is utilized in methods described herein. However, such background fluorescence is contemplated to be differentiable from a signal state and as such nucleotide incorporation of an unlabeled nucleotide (or “dark” nucleotide) is still discernible.

In one example of the method described herein, “A” nucleotide conjugate is determined by a signal state in the first imaging event and a dark state in the second imaging event; “C” nucleotide conjugate is determined by a dark state in the first imaging event and a signal state in the second imaging event; “T” nucleotide conjugate is determined by a signal state in the first imaging event and a signal state in the second imaging event; and “G” nucleotide conjugate is determined by a dark state in the first imaging event and a dark state in the second imaging event.

In some embodiments, the method further comprises contacting the extended copy polynucleotides with a chemical reagent to remove the first label of the first type of nucleotide after the first fluorescent measurement and prior to the second fluorescent measurement. In further embodiments, the first label is covalently attached to the nucleobase of the first type of nucleotide via a cleavable linker comprising an azido, allyl, or disulfide moiety, or any other suitable moiety as described herein. In some embodiments, the chemical reagent for removing the first label of the first type of nucleotide comprises tris(hydroxypropyl)phosphine (THP). In further embodiments, the chemical reagent to remove the first label is in the same composition as the labeled avidin. In other embodiments, the chemical reagent to remove the first label is in a separate composition from the labeled avidin.

In some embodiments of the sequencing method described herein, the method further comprises (vi) removing the 3′ blocking group of the incorporated nucleotide prior to the next sequencing cycle. In some such embodiments, the label and the 3′ blocking group are removed in a single step (e.g., under the same chemical reaction condition). In other embodiments, the label and the 3′ blocking group are removed in two separate steps (e.g., the label and the 3′ blocking group are removed under two separate chemical reaction conditions). In some further embodiments, a post cleavage washing step (vii) is used after the label and the 3′ blocking group are removed.

In some embodiments, the imaging steps (iii) and (v) are performed by a single light source (e.g., a laser). In particular, the light source may have a wavelength between about 450 nm to about 460 nm, or between about 510 nm to about 540 nm, or between about 520 nm to about 535 nm. The sequencing method described herein is also referred to as one-channel sequencing. In further embodiments, the fluorescent measurements of the emissions are performed by CMOS based single wavelength detection at about 570 nm and above.

In further embodiments of the sequencing method described herein, steps (ii) through (vi) are performed in repeated cycles (e.g., at least 30, 50, 100, 150, 200, 250, 300, 400, or 500 times) and the method further comprises sequentially determining the sequence of at least a portion of the target polynucleotide based on the identity of each sequentially incorporated nucleotide conjugates. In some such embodiments, steps (ii) through (vi) or (ii) through (vii) are repeated at least 50 cycles. In some such embodiments, the four different types of nucleotide conjugates are simultaneously present and compete for incorporation during each cycle. In some further embodiments, the incorporation of the nucleotide conjugates is performed by a polymerase (e.g., a DNA polymerase). Exemplary polymerases include but not limited to Pol 812, Pol 1901, Pol 1558 or Pol 963. The amino acid sequences of Pol 812, Pol 1901, Pol 1558 or Pol 963 DNA polymerases are described, for example, in U.S. Patent Publication Nos. 2020/0131484 A1 and 2020/0181587 A1, both of which are incorporated by references herein.

In some embodiments of the method described herein, the nucleotide conjugates in the mixture in step (ii) comprise nucleotide types selected from the group consisting of A, C, G, T and U, and non-natural nucleotide analogs thereof. In further embodiments, the mixture in step (ii) comprises four different types of nucleotide conjugates (A, C, G, and T or U), or non-natural nucleotide analogs thereof. In further embodiments, the four different types of nucleotide conjugates are dATP, dCTP, dGTP and dTTP or dUTP, or non-natural nucleotide analogs thereof. In further embodiments, each of the four types of nucleotide conjugates in the incorporation mixture contains a 3′ blocking group. In some further embodiments, the first label, the second label, and the one or more fluorescent moieties of the avidin in the composition are identical. In other embodiments, the first label, the second label, and the one or more fluorescent moieties of the avidin in the composition are different by can be excited by the same light source and emits a signal that can be collected in the same detection channel.

A further aspect of the present disclosure relates to another method for determining the sequences of a plurality of different target polynucleotides in parallel, comprising:
(i) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(ii) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein one type of nucleotides is the labeled avidin nucleotide conjugate described herein, and each of the four types of nucleotides comprises a 3′ blocking group;
(iii) imaging and performing one or more fluorescent measurements of the extended copy polynucleotides; and
(iv) removing the 3′ blocking group of the incorporated nucleotides.

In some embodiments of the alternative sequencing method described herein, the imaging step (iii) uses two light sources operating at different wavelengths (e.g., at 450-460 nm and 520-535 nm), and the aqueous solution of step (ii) comprises a first type of labeled avidin nucleotide conjugate (with first labels), a second type of labeled nucleotide (with a second label), a third type of labeled nucleotide (may be labeled with both the first label and the second label), and a fourth type of unlabeled nucleotide. In some other embodiments of the alternative sequencing method described herein, the imaging step (iii) uses four light sources operating at different wavelengths, and the aqueous solution of step (ii) comprises a first type of labeled avidin nucleotide conjugate, a second type of labeled nucleotide, a third type of labeled nucleotide, and a fourth type of labeled nucleotide, and the four types of labeled nucleotides are spectrally distinguishable. In some embodiments, the method further comprises a post-cleavage wash step (v). In some embodiments, steps (ii) through (iv) or (ii) through (v) are repeated at least 50, 100, 150, 200, 250, 300, 350, 400, 450 or 500 cycles. In some embodiments, the four types of nucleotides comprise dATP, dCTP, dGTP and dTTP or dUTP, or non-natural nucleotide analogs thereof.

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxy group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′ blocking group and fluorescent dye compounds can then be removed (deprotected) (simultaneously or sequentially) to expose the nascent chain for further nucleotide incorporation.

The method, as exemplified above, utilizes the incorporation of fluorescently labeled, 3′-blocked nucleotides A, G, C, and T into a growing strand complementary to the immobilized polynucleotide, in the presence of DNA polymerase. The polymerase incorporates a base complementary to the target polynucleotide but is prevented from further addition by the 3′-blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′ hydroxy group that serves as a primer or initiation point for the addition of further nucleotides in the sequencing reaction. The region of the template to be sequenced will overhang this free 3′ hydroxy group on the complementary strand. The overhanging region of the template to be sequenced may be single stranded but can be double-stranded, provided that a “nick is present” on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxy group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO0157248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxy group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in W02005/047301 (incorporated herein by reference). A still further example of solid-supported template polynucleotides is where the template polynucleotides are attached to hydrogel supported upon silica-based or other solid supports, for example, as described in WO00/31148, WO01/01143, WO02/12566, WO03/014392, U.S. Pat. No. 6,465,178 and WO00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO00/06770 and WO 01/57248, each of which is incorporated herein by reference. Although one use of the labeled nucleotides of the disclosure is in sequencing-by-synthesis reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotides labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

### EXAMPLES

Additional embodiments are disclosed in further detail in the following examples, which are not in any way intended to limit the scope of the claims.

### Example 1

### Fluorescent Signal Intensity Comparison in Solution

In this example, streptavidin was randomly labeled with fluorescent dyes, forming an amide bond between the amino group of lysine on the streptavidin surface and the carboxylic acid group on the dye. Solution data revealed that the fluorescently labeled streptavidin produced one-half of the signal intensity compared to a solution of the dye itself. In the other words, one fluorescently labeled streptavidin molecule was three times brighter than one dye molecule in the solution, although on average there are six dyes per streptavidin. FIG. 2A illustrates the relative fluorescent signal intensity in solution, comparing one fluorescent dye molecule and one multi-dye labeled streptavidin molecule. In particular, the solution data compared the fluorescent intensity of same molar ratio of a known green dye NR550C4 versus streptavidin labeled with six NR550C4. UV-spectra quantification confirmed there was six NR550C4 covalently attached to the streptavidin. In an ideal scenario, 6 times of signal intensity than a single molecule of NR550C4 should be expected but only 3 times of intensity was observed.

### Example 2

### Fluorescent Signal Intensity Comparison on Flow Cell

FIG. 2B is a one-dye SBS scatterplot generated from a modified sequencing run on MiSeg™. The incorporation mix included dark ffC comprising a biotin moiety. Both ffA and ffT were labeled with a green dye NR550S0, with ffA also contained a disulfide linker between the nucleobase and the label; ffG is unlabeled. After taken the first image, a composition including six-dye labeled streptavidin and THP was introduced to label ffC and cleave the label from ffA. Then a second image was taken. It was observed that ffC labeled with six-dye streptavidin only resulted in only equivalent signal generated by ffT.

### Example 3

### Titration of Fully Functionalized Cytosine with Varying Ratios of Biotin and Streptavidin

In this example, the SBS was conducted on a blue/green MiSeq™. First, biotin (in the form of ffC-biotin) was incubated with Strep-PEG12-Coumarin Dye A in various molar ratios. Specifically, molar ratios of 1:1, 2:1, 3:1, 4:1 and 6:1 ffC-biotin:Strep-PEG12-Coumarin Dye A were pre-incubated in Streptavidin binding buffer (10 mM tris pH 7.5, 1M NaCl, 0.5 mM EDTA, 0.05% Tween20) at room temp for 5 min prior to testing on the MiSeg™. The ffN set used in the SBS were: ffA-Coumarin Dye B/ffA-NR550S0 (1:1 molar ratio), ffC-biotin, dark G, ffT-AF550POPOS0.

Coumarin dye A is disclosed in U.S. Publication No. 2022/0033900 A1, having the structure moiety

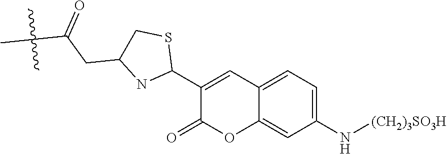

when conjugated with streptavidin through the PEG linker. Coumarin dye B is disclosed in U.S. Publication No. 2020/0277670 A1, having the structure moiety

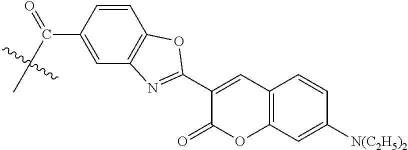

when conjugated with the ffA. AF550POPOS0 is disclosed in U.S. Publication No. 2018/0282791 A1, having the structure moiety

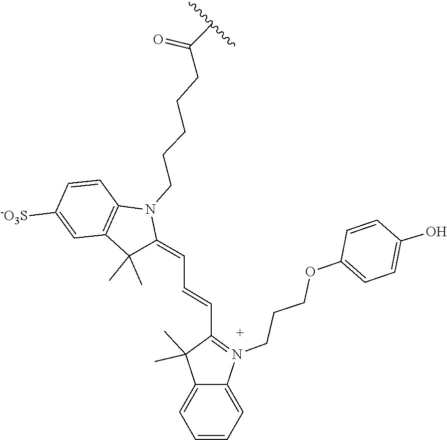

when conjugated with the ffT.

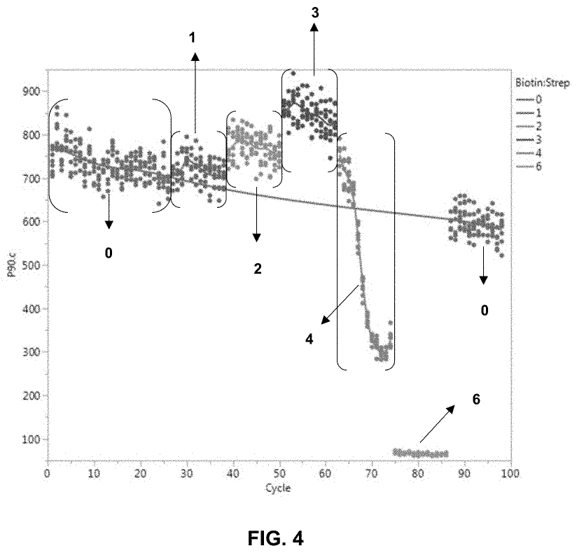

FIG. 4 illustrates the blue signal intensity as a function of the number of SBS cycles to determine the effect of titrating ffC-biotin into Strep-PEG12-Coumarin Dye A prior to SBS. Streptavidin has four biotin binding sites. The experiment showed that by preincubating ffC-biotin with Strep-PEG12-Coumarin Dye A at 1:1, 2:1 or 3:1 molar ration, there was an increase in signal related to the increasing biotin:Strep ratio. Without being bound by a particular theory, the increase in signal might be attributed to an increasing number of Strep-PEG12-Coumarin Dye A entities binding to the polynucleotide strands which incorporated ffC-biotin. Due to the blocking of the available sites on Strep-PEG12-Coumarin Dye A by ffC-biotin, it allowed for each Strep-PEG12-Coumarin Dye A to bind to an individual polynucleotide that incorporated ffC-biotin, rather than to crosslink (or bind to) two or more polynucleotide strands that incorporated ffC-biotin.

SBS began to fail at a ratio of 4:1 and failed completely at 6:1 biotin:Strep, suggesting that all biotin binding sites of streptavidin were blocked and there were no available sites on the streptavidin for binding with the incorporated ffC-biotin. In summary, sequencing data revealed an increased signal detection in circumstances where biotin and streptavidin were premixed in ratios of 1:1, 2:1 and 3:1. Best signal detection was observed where biotin and streptavidin were pre-mixed in a 3:1 ratio.

1. A labeled avidin for polynucleotide sequencing, wherein the avidin is labeled with one or more fluorescent dye moieties, wherein at least one biotin-binding site of the avidin is blocked with a biotin moiety-containing molecule or an analog thereof, and wherein the labeled avidin comprises at least one open biotin-binding site.

2. The labeled avidin of claim 1, wherein one biotin-binding site of the avidin is blocked with the biotin moiety-containing molecule or the analog thereof.

3. The labeled avidin of claim 1, wherein two biotin-binding sites of the avidin are blocked with the biotin moiety-containing molecule or the analog thereof.

4. (canceled)

5. The labeled avidin of claim 1, wherein the avidin is labeled with two to eight fluorescent dye moieties.

6.-8. (canceled)

9. The labeled avidin of claim 1, wherein the fluorescent dye moieties are excitable by a light source having a wavelength between 400 nm to 650 nm.

10. (canceled)

11. The labeled avidin of claim 1, wherein the avidin is streptavidin.

12. The labeled avidin of claim 1, wherein the biotin moiety-containing molecule or the analog thereof comprises a biotin moiety that is covalently attached to a nucleotide or nucleoside.

13. The labeled avidin of claim 1, wherein the biotin moiety-containing molecule or the analog thereof is a free biotin molecule or an analog that is not covalently attached to a nucleotide or nucleoside.

14. A labeled avidin nucleotide conjugate, comprising an avidin-binding partner covalently attached to the nucleotide, optionally via a cleavable linker, wherein the avidin-binding partner is conjugated with the labeled avidin of claim 1 via non-covalent interaction.

15. (canceled)

16. The labeled avidin nucleotide conjugate of claim 14, wherein the avidin-binding partner is covalently attached to the nucleobase of the nucleotide via a cleavable linker.

17. The labeled avidin nucleotide conjugate of claim 14, wherein the cleavable linker comprising an azido, an allyl, or a disulfide moiety.

18. The labeled avidin nucleotide conjugate of claim 14, wherein nucleotide conjugate comprises a 3′ hydroxy blocking group.

19. An oligonucleotide or polynucleotide conjugate, comprising an avidin conjugated to a terminal nucleotide moiety of the oligonucleotide or polynucleotide, wherein the avidin is labeled with one or more fluorescent dye moieties, and at least one biotin-binding site of the avidin is blocked with a biotin moiety-containing molecule or an analog thereof, wherein the terminal nucleotide moiety comprises an avidin binding partner covalently attached thereto, and the labeled avidin is conjugated to the avidin binding partner of the terminal nucleotide moiety by non-covalent interaction.

20. The oligonucleotide or polynucleotide conjugate of claim 19, wherein one, two or three biotin-binding sites of the avidin is blocked with the biotin moiety-containing molecule or the analog thereof.

21.-22. (canceled)

23. The oligonucleotide or polynucleotide conjugate of claim 19, wherein the terminal nucleotide moiety comprises a 3′ hydroxy blocking group, and the avidin binding partner comprises a biotin moiety covalently attached to the nucleobase of the terminal nucleotide moiety, optionally through a cleavable linker, and wherein the avidin is conjugated to the terminal nucleotide moiety through non-covalent interaction between the biotin moiety and a biotin-binding site of the avidin.

24. The oligonucleotide or polynucleotide conjugate of claim 19, wherein the oligonucleotide or polynucleotide conjugate is at least partially complementary and hybridized to a target polynucleotide immobilized on a surface of a solid support.

25. The oligonucleotide or polynucleotide conjugate of claim 19, wherein the avidin is streptavidin.

26.-30. (canceled)

31. A method for determining the sequences of a plurality of different target polynucleotides in parallel, comprising:
(i) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(ii) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein a first type of nucleotide comprises a first label, a second type of nucleotide comprises a second label, a third type of nucleotide is unlabeled and functionalized with an avidin binding partner, a fourth type of nucleotide is not labeled, and each of the four types of nucleotides comprises a 3′ hydroxy blocking group;
(iii) performing a first fluorescent measurement of the extended copy polynucleotides;
(iv) contacting the extended copy polynucleotides with a labeled avidin according to claim 1; and
(v) performing a second fluorescent measurement of the extended copy polynucleotides.

32. (canceled)

33. The method of claim 31, further comprising contacting the extended copy polynucleotides with a chemical reagent to remove the first label of the first type of nucleotide after the first fluorescent measurement and prior to the second fluorescent measurement.

34.-43. (canceled)

44. A method for determining the sequences of a plurality of different target polynucleotides in parallel, comprising:
(i) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(ii) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein one type of nucleotides is the labeled avidin nucleotide conjugate of any one of claim 14, and each of the four types of nucleotides comprises a 3′ hydroxy blocking group;
(iii) performing one or more fluorescent measurements of the extended copy polynucleotides; and
(iv) removing the 3′ hydroxy blocking group of the incorporated nucleotides.

45.-48. (canceled)

49. A kit for polynucleotide sequencing, comprising:
a labeled avidin according to claim 1.

50. The kit of claim 49, further comprising an incorporation composition comprising a first type of nucleotide having a first label, a second type of nucleotide having a second label, a third type of nucleotide is unlabeled and functionalized with an avidin binding partner, a fourth type of nucleotide is not labeled, and each of the four types of nucleotides comprises a 3′ hydroxy blocking group.

51. The kit of claim 50, wherein the avidin is streptavidin and the avidin binding partner comprises a biotin moiety covalently attached to the nucleobase of the third type of nucleotide, optionally through a cleavable linker.

52. The kit of claim 50, wherein the first label is covalently attached to the nucleobase of the first type of nucleotide via a cleavable linker comprising an azido, allyl, or disulfide moiety.

53. The kit of claim 50, further comprising a cleavage composition comprising a chemical reagent capable of removing the first label of the first type of nucleotide.

54. The kit of claim 53, wherein the chemical reagent for removing the first label of the first type of nucleotide comprises tris(hydroxypropyl)phosphine (THP).

55. (canceled)

56. A kit for polynucleotide sequencing, comprising a labeled avidin nucleotide conjugate according to claim 14.
